(19) United States
(12) Patent Application Publication
(10) Pub. No.: US 2016/0166164 A1
(43) Pub. Date: Jun. 16, 2016

(54) Method and Apparatus for Detecting Neural Injury

(71) Applicant: Saluda Medical Pty Limited, Artarmon (AU)

(72) Inventors: Milan Obradovic, Artarmon (AU); Tracy Cameron, Artarmon (AU); Robert Bruce Gorman, Artarmon (AU); Nastaran Hesam Shariati, Artarmon (AU); John Louis Parker, Artarmon (AU)

(73) Assignee: Saluda Medical Pty Limited

(21) Appl. No.: 14/954,529

(22) Filed: Nov. 30, 2015

(30) Foreign Application Priority Data
Dec. 11, 2014 (AU) AU2014905032

(51) Int. Cl. A61B 5/04; A61B 5/00

(52) U.S. Cl. CPC A61B 5/04001; A61B 5/7282; A61B 5/4836; A61B 5/686; A61B 5/407

(57) ABSTRACT

An implantable device for monitoring for neural injury has a plurality of electrodes including stimulus electrodes and sense electrodes. A stimulus source provides a stimulus to be delivered to a nerve in order to give rise to an evoked action potential. Measurement circuitry records a neural compound action potential signal sensed at the sense electrodes. A stream of control stimuli are applied to the nerve over time and the evoked neural responses are measured. A diagnostic parameter of the measured neural responses is monitored over time, in order to detect a change in the diagnostic parameter. If a change in the diagnostic parameter occurs over time, an indication is output that neural injury has occurred.

### TECHNICAL FIELD

The present invention relates to monitoring compound action potentials during surgery to assist with implantable electrode placement, and/or to monitoring compound action potentials post surgery to monitor for neural damage and/or onset of neural trauma.

### BACKGROUND OF THE INVENTION

There are a range of situations in which it is desirable to apply neural stimuli in order to give rise to a compound action potential (CAP). For example, neuromodulation is used to treat a variety of disorders including chronic pain, Parkinson's disease, and migraine. A neuromodulation system applies an electrical pulse to tissue in order to generate a therapeutic effect. When used to relieve chronic pain, the electrical pulse is applied to the dorsal column (DC) of the spinal cord. Such a system typically comprises an implanted electrical pulse generator, and a power source such as a battery that may be rechargeable by transcutaneous inductive transfer. An electrode array is connected to the pulse generator, and is positioned in the dorsal epidural space above the dorsal column. An electrical pulse applied to the dorsal column by an electrode causes the depolarisation of neurons, and generation of propagating action potentials. The fibres being stimulated in this way inhibit the transmission of pain from that segment in the spinal cord to the brain. To sustain the pain relief effects, stimuli are applied substantially continuously, for example at 100 Hz.

Neuromodulation may also be used to stimulate efferent fibres, for example to induce motor functions. In general, the electrical stimulus generated in a neuromodulation system triggers a neural action potential which then has either an inhibitory or excitatory effect. Inhibitory effects can be used to modulate an undesired process such as the transmission of pain, or to cause a desired effect such as the contraction of a muscle.

The typical procedure for placing surgical leads for a spinal cord stimulator (SCS) requires a laminectomy and placing the patient under general anaesthesia. Complications of implanting a surgical lead can include damage to the spinal cord due to direct pressure of the lead as it is placed into the epidural space, cerebrospinal fluid (CSF) leakage, damage to the dorsal roots, and infection of the battery or electrode site, any of which can cause paralysis, weakness, or chronic pain. Post-operative damage can also occur due to the development of a hematoma over the lead which then creates pressure on the lead and damages the dorsal column axons, or due to electrical stimulation at an excessive current amplitude and/or frequency. Although such intraoperative and postoperative complications are not common, they can be very serious. Pressure applied to the spinal cord during surgery while the patient is under general anesthetic can have serious and long lasting effects and may not be noticed until after a procedure. Currently, the only way neural damage is assessed is by having a patient provide feedback during the procedure, which requires the patient to be awake during these periods.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood that the element may be any one of the listed options, or may be any combination of two or more of the listed options.

### SUMMARY OF THE INVENTION

According to a first aspect the present invention provides a method of monitoring for neural injury, the method comprising:
delivering a stream of control stimuli to a neural pathway over time;
measuring neural responses evoked by the control stimuli over time;
monitoring a diagnostic parameter of the measured neural responses over time, in order to detect a change in the diagnostic parameter, and
if a change in the diagnostic parameter occurs over time, outputting an indication that neural injury has occurred.

According to a second aspect the present invention provides an implantable device for monitoring for neural injury, the device comprising:
a plurality of electrodes including one or more nominal stimulus electrodes and one or more nominal sense electrodes;
a stimulus source for providing a stimulus to be delivered from the one or more stimulus electrodes to a neural pathway in order to give rise to an evoked action potential on the neural pathway;
measurement circuitry for recording a neural compound action potential signal sensed at the one or more sense electrodes; and
a control unit configured to:
   deliver a stream of control stimuli to the neural pathway over time;
   measure neural responses evoked by the control stimuli over time;
   monitor a diagnostic parameter of the measured neural responses over time, in order to detect a change in the diagnostic parameter, and
   if a change in the diagnostic parameter occurs over time, output an indication that neural injury has occurred.

According to a third aspect the present invention provides a non-transitory computer readable medium for monitoring for neural injury, comprising the following instructions for execution by one or more processors:
computer program code means for delivering a stream of control stimuli to a neural pathway over time;
computer program code means for measuring neural responses evoked by the control stimuli over time;
computer program code means for monitoring a diagnostic parameter of the measured neural responses over time, in order to detect a change in the diagnostic parameter, and
computer program code means for, if a change in the diagnostic parameter occurs over time, outputting an indication that neural injury has occurred.

The present invention thus recognises that the compound action potential can be used to monitor for damage occurring to the neural pathway over time, and can in some embodiments be used to provide an essentially immediate diagnostic to rapidly detect the onset of the effects of neural injury.

In some embodiments of the invention the control stimuli are delivered, and the diagnostic ECAP parameter is monitored, substantially continuously. Such embodiments tray be performed throughout a surgical procedure, or throughout a postoperative period of interest, so that any change in the diagnostic parameter can be noted in substantially real time, to maximise the opportunity to prevent further injury before it become irreversible.

The diagnostic parameter may in some embodiments comprise ECAP amplitude. For example a reduction in ECAP amplitude may be taken as a diagnostic indicator of neural injury. Additionally or alternatively, the diagnostic parameter may in some embodiments comprise ECAP conduction velocity, whereby a reduction in conduction velocity may be taken as a diagnostic indicator of neural injury. Additionally or alternatively, the diagnostic parameter may in some embodiments comprise ECAP peak latency such as the N1 or P1 peak latency, whereby an increase in ECAP peak latency may be taken as a diagnostic indicator of neural injury. Additionally or alternatively, the diagnostic parameter may be the existence of ECAPs with multiple peaks consistent with spontaneous firing. Additionally or alternatively, the diagnostic parameter may be the emergence of a late response during the period around 2-5 ms after ECAP onset.

The diagnostic indicator may be observed over multiple stimulus-measurement cycles, so that a curve of the diagnostic indicator can be identified from multiple measurement points. In such embodiments an observed change in the diagnostic indicator from a first steady state and involving an exponential or asymptotic or other decay towards a second steady state over the course of seconds or minutes, may be taken as particularly indicative of neural damage.

The control stimuli may also serve as therapeutic stimuli. Alternatively the control stimuli may be of a different morphology to therapeutic stimuli, the control stimuli morphology being configured to produce ECAPs which are sensitive to the effects of neural injury, and such control stimuli may be interposed between a sequence of therapeutic stimuli.

The method of the present invention may be performed during implantation of an electrode lead during surgery, whereby electrodes of the electrode lead being implanted serve as both the stimulus and sense electrodes, so as to continuously intraoperatively monitor the spinal cord or nerve for damage. In such embodiments, the indication of neural injury is preferably output in a manner which can be promptly perceived by the implanting surgeon, such as an audible alarm. Additionally or alternatively the method of the present invention may in some embodiments be performed post operatively to alert treating physicians and/or the implant recipient to damage which is or has occurred to the spinal cord or nerve, as may result from some damaging process such as increasing pressure due to hematoma. In such embodiments the indication of neural injury may comprise a text message to the patient advising them to minimise physical activity and promptly contact their physician, or a message may be transmitted from the implant to an external device and thence via any suitable communications channel directly to a physician.

Moreover, present invention recognises that different modes of neural injury manifest differently in the effects which occur on the ECAPs. Accordingly, in some embodiments of the present invention a plurality of diagnostic indicators are monitored for change, or for emergence, and the indication that a neural injury has occurred also contains an indication of a likely mode of injury. Such embodiments may hasten diagnosis and treatment of the neural injury, which can be of much importance in cases where the cause of the neural injury will otherwise lead to irreversible neural damage if not promptly identified and treated.

The stimulus and sense electrodes may in some embodiments be configured so that ECAP propagation is in an orthodromic direction from the stimulus electrodes to the sense electrodes. Additionally or alternatively, the stimulus and sense electrodes may in some embodiments be configured so that ECAP propagation is in an antidromic direction from the stimulus electrodes to the sense electrodes.

The control stimuli could simply be a sequence of stimuli which are each of the same morphology, i.e of unchanged amplitude, pulse width, and the like, so that in the absence of neural injury or electrode movement, a substantially constant ECAP will be evoked by each of the stimuli, so that changes in the ECAP occurring over time can be more clearly taken to be indicative of neural injury. Alternatively the control stimuli may comprise any sequence or sequences of differing stimuli configured which allow the diagnostic parameter of the measured ECAPs to be satisfactorily distinguished over time.

Some embodiments may provide compensation for electrode movement, so that ECAP amplitude variations resulting from changes in the electrode-to-nerve distance can be discounted from considerations of whether neural injury has occurred. Alternative embodiments may instead focus on non-amplitude related parameters such as latency and velocity.

The present invention thus provides for the use of evoked compound action potentials recorded directly from a neurostimulator lead to provide a direct measure of the neurophysiological responses and to obtain a direct indication of the onset of neurological damage.

### BRIEF DESCRIPTION OF THE DRAWINGS

An example of the invention will now be described with reference to the accompanying drawings, in which:

FIG. 1 schematically illustrates an implanted spinal cord stimulator;

FIG. 2 is a block diagram of the implanted neurostimulator;

FIG. 3 is a schematic illustrating interaction of the implanted stimulator with a nerve;

FIG. 4 illustrates the stimulus and measurement of ECAPs in order to detect neural damage;

FIG. 5 illustrates ECAP recordings obtained from below a damage site, during a period in which differing levels of trauma were intermittently applied to the nerve;

FIG. 6 illustrates an extract of selected recordings from those reflected in FIG. 5, showing the reduction in amplitude and conduction velocity of the ECAP which occurs over time in response to neural damage;

FIG. 7 illustrates recordings obtained at ten second intervals on four electrodes, showing the reduction in amplitude and conduction velocity of the ECAP which occurs over time in response to neural damage;

FIG. 8 illustrates ECAP recordings obtained from below a damage site, both before and after trauma was applied to the nerve, including an extract of selected recordings showing the reduction in amplitude and conduction velocity of the ECAP which occurs over time in response to neural damage;

FIGS. 9a and 9b shows a reduction in amplitude is a key diagnostic of neural damage;

FIG. 10 shows that a reduction in conduction velocity is a further diagnostic of neural damage;

FIG. 11 shove that an increase in the latency of the ECAP second peak is a diagnostic of neural damage;

FIG. 12 is a computational model of membrane voltage over time for increasing length of the node of Ranvier; and

FIG. 13 is a computational model of the conduction velocity versus the length of the node of Ranvier.

### DESCRIPTION OF THE PREFERRED EMBODIMENTS

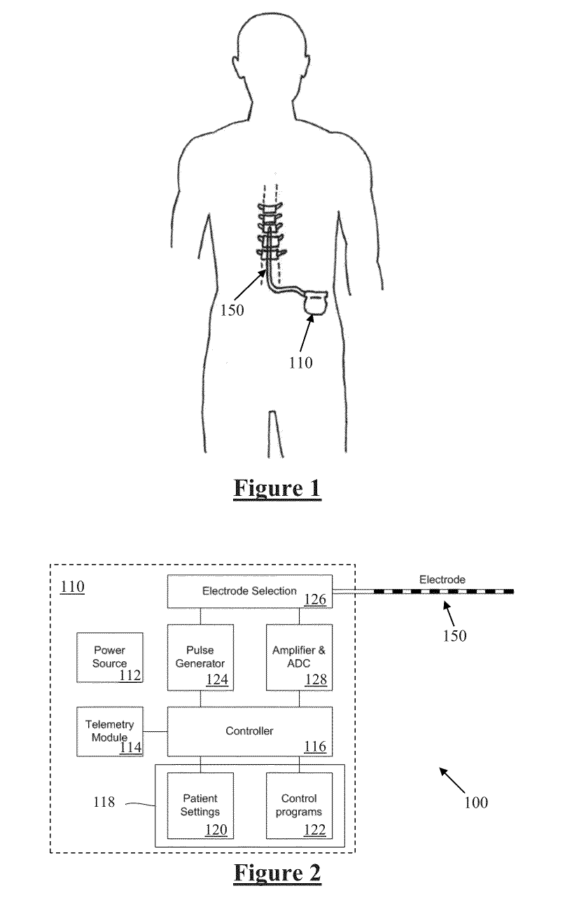

FIG. 1 schematically illustrates an implanted spinal cord stimulator 100. Stimulator 100 comprises an electronics module 110 implanted at a suitable location in the patient's lower abdominal area or posterior superior gluteal region, and an electrode assembly 150 implanted within the epidural space and connected to the module 110 by a suitable lead.

FIG. 2 is a block diagram of the implanted neurostimulator 100. Module 110 contains a battery 112 and a telemetry module 114. In embodiments of the present invention, any suitable type of transcutaneous communication, such as infrared (IR), electromagnetic, capacitive and inductive transfer, may be used by telemetry module 114 to transfer power and/or data between an external device and the electronics module 110.

Module controller 116 has an associated memory 118 storing patient settings 120, control programs 122 and the like. Controller 116 controls a pulse generator 124 to generate stimuli in the form of current pulses in accordance with the patient settings 120 and control programs 122. Electrode selection module 126 switches the generated pulses to the appropriate electrode(s) of electrode array 150, for delivery of the current pulse to the tissue surrounding the selected electrode. Measurement circuitry 128 is configured to capture measurements of neural responses sensed at sense electrode(s) of the electrode array as selected by electrode selection module 126.

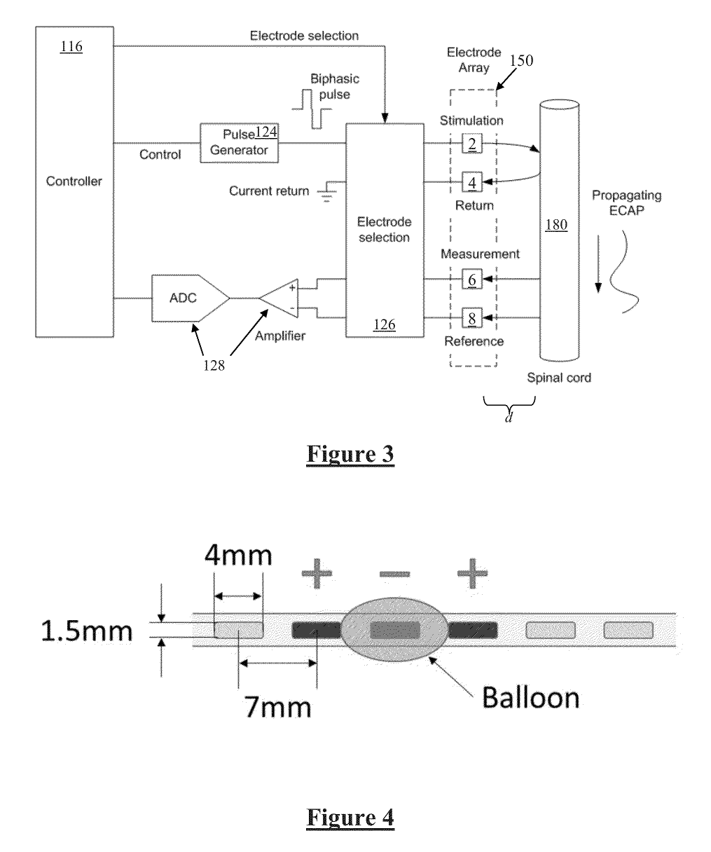

FIG. 3 is a schematic illustrating interaction of the implanted stimulator 100 with a nerve 180, in this case the spinal cord however alternative embodiments may be positioned adjacent any desired neural tissue including a peripheral nerve, visceral nerve, parasympathetic nerve or a brain structure. Electrode selection module 126 selects a stimulation electrode 2 of electrode array 150 to deliver an electrical current pulse to surrounding tissue including nerve 180, and also selects a return electrode 4 of the array 150 for stimulus current recovery to maintain a zero net charge transfer.

Delivery of an appropriate stimulus to the nerve 180 evokes a neural response comprising a compound action potential which will propagate along the nerve 180 as illustrated, for therapeutic purposes which in the case of spinal cord stimulator for chronic pain might be to create paraesthesia at a desired location.

The device 100 is further configured to sense the existence and intensity of compound action potentials (CAPs) propagating along nerve 180, whether such CAPs are evoked by the stimulus from electrodes 2 and 4, or otherwise evoked. To this end, any electrodes of the array 150 may be selected by the electrode selection module 126 to serve as measurement electrode 6 and measurement reference electrode 8. Signals sensed by the measurement electrodes 6 and 8 are passed to measurement circuitry 128, which for example may operate in accordance with the teachings of International Patent Application Publication No. WO2012155183 by the present applicant, the content of which is incorporated herein by reference.

With any electrode design the surgical approach and placement of the lead brings the risk of damage to the spinal cord. Pressure or mechanical damage can occur at the time of surgical placement or at some point after the lead is placed as the result of a haematoma. The following embodiments illustrate how ECAP signals can be used during and after surgery to monitor the cord for signs of pressure or mechanical damage.

The effect of pressure on the spinal cord during spinal cord stimulation was investigated in an animal model. We examined the effectiveness of monitoring the ECAP signal during lead implantation in identifying the onset of neural injury. Preliminary data was collected first in an acute sheep model. This animal model was chosen due to its size, which allowed the use of human-sized leads. It also allowed the evaluation of tissue after the study. As shown in FIG. 4 a custom SCS paddle lead with 24 electrodes, 4 mm long and 1.5 mm wide, and spanning 182 mm (7 mm center to center spacing) was implanted into the epidural space of three sheep. The lead was connected to a stimulation and recording system. Baseline ECAP signals were recorded to allow calculation of various neural properties. A compliant balloon catheter was fixed to the dorsal side at the 12^{th} electrode which, when inflated, caused the balloon to exert pressure on the spinal cord at that position. The balloon (2 mL volume) was inflated with air to selected volumes using the syringe provided for the catheter.

Tripolar, biphasic stimulation, at a constant current (approximately one and a half times the threshold current), was delivered at the top, middle (on the electrode with the balloon opposite it) and the bottom of the lead. ECAPs that had propagated through and from the pressure site were measured.

Constant pressure was applied for approximately five minutes and then released for five minutes while ECAPs were continuously monitored on the sense electrodes. The volume was increased after each release period until the ECAP did not recover within 5 minutes. ECAPs were examined for any changes from baseline. Affected tissue was removed from the cord and examined histologically.

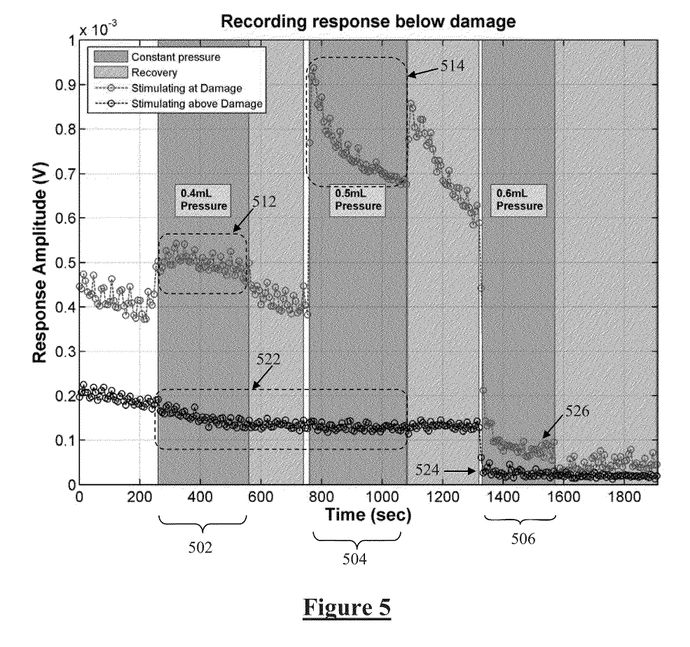

FIG. 5 shows recordings obtained from below the damage site, i.e. from electrodes which only received ECAPs if they had travelled past the damage site. Applying low volumes of air to the catheter (<0.4 mL) did not have any noticeable effects on the ECAP properties. At a threshold volume (0.4 ml) the ECAP amplitude 512 evoked by stimuli delivered at the site of the damage increased during the period 502 for which the pressure was applied, and then decreased over time to return substantially to the original steady state level. Upon the application of 0.5 of air for the duration of the time period 504, the ECAP amplitude 514 evoked by stimuli delivered at the site of the damage increased during the period 504, and then decreased over time but did not reach a new steady state before the subsequent trauma was applied. During periods 502 and 504, stimuli were also applied above the damage site, and the evoked ECAPs therefrom were detected below the damage site. These ECAP amplitudes 522 did not appreciably alter during time periods 502 and 504. However, at a critical volume (0.6 ml) a trauma can clearly be detected in the observed ECAPs, as the ECAP amplitude dropped when propagating through the pressure site (524) in both the orthodromic and antidromic direction, and the shape of the signal changed. ECAPs 526 propagating from the location of the pressure also diminished. After the pressure was released the ECAP amplitude did not return to baseline within 5 minutes.

Histological evaluation revealed damage consistent with ischemia. The electrophysiological responses were altered after irreversible damage; amplitude of responses dropped, conduction velocity decreased and the remaining responding fibres produced ECAPS with multiple peaks consistent with spontaneous firing. Data indicates irreversible damage to the fibres where the pressure was applied, preventing axons from being recruited and ECAPs propagating through the site. Electrophysiological responses to damage include the amplitude of responses dropping, conduction velocity decreasing, and the remaining responding fibres produced ECAPS with multiple peaks consistent with spontaneous firing, any or all of which may thus present useful diagnostic indicators for neural damage in various embodiments of the present invention.

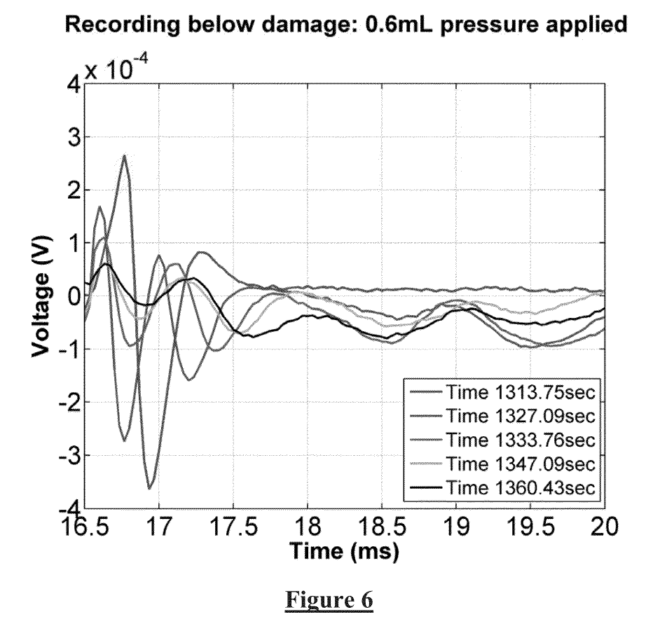

FIG. 6 shows recordings obtained from below the damage site prior to and during the period 506 in which 0.6 of pressure was applied. As can be seen, over time the ECAPs observed reduce in amplitude and exhibit multiple later responses during the time period around 2-5 ms after ECAP onset, consistent with spontaneous firing, and these characteristics may thus be used to detect neural damage by observing such ECAP parameters over time.

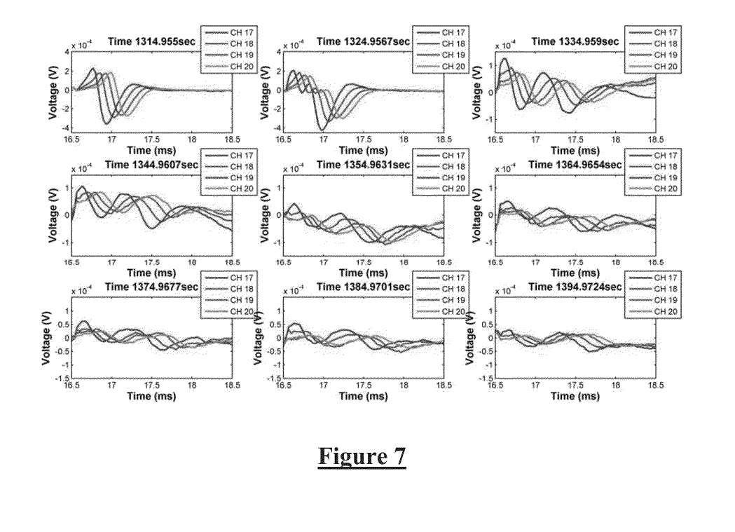

FIG. 7 presents recordings from multiple electrodes at various times prior to and during period 506 shown in FIG. 5. Such multi-electrode recordings permit analysis of conduction velocity as a diagnostic indicator of damage.

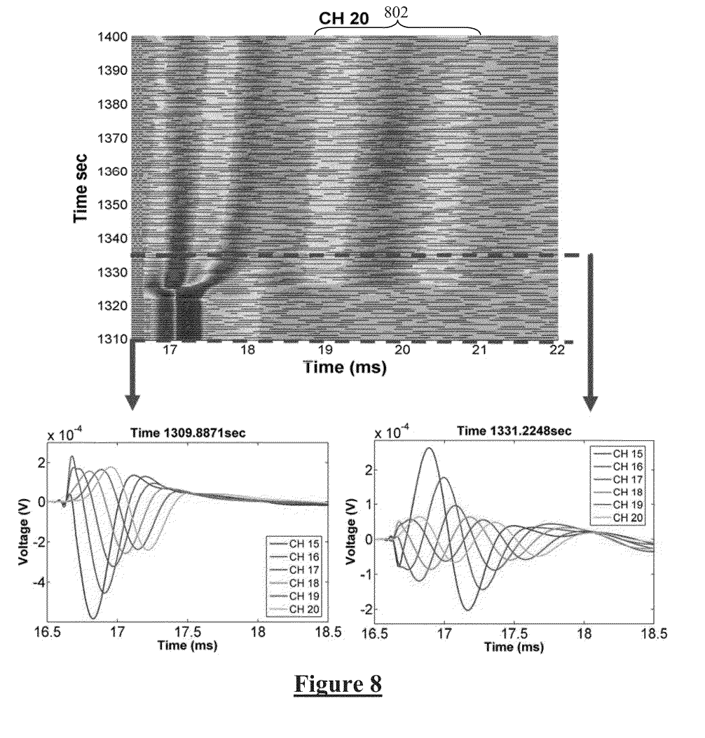

FIG. 8 illustrates ECAP recordings obtained from below a damage site, both before and after trauma was applied to the nerve, including respective extracts of selected recordings showing the reduction in amplitude and conduction velocity of the ECAP which occurs over time in response to neural damage, and the emergence of a late response during the time period 802 around 2-5 ms after ECAP onset.

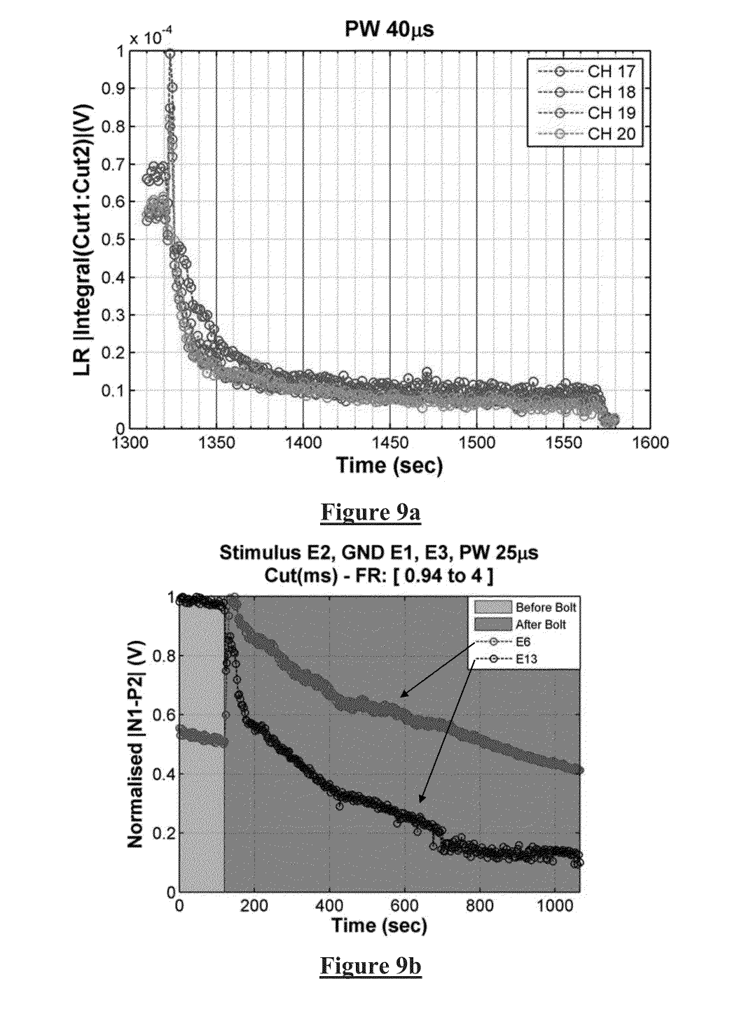

FIG. 9a shows that a reduction in amplitude is a key diagnostic of the neural damage which occurred at the onset of time period 506. FIG. 9b shows data from another investigation, in which ECAP amplitude was observed either side of an injury site. Electrode E6, which is in the same side of the injury site as a stimulus electrode, returns approximately to the pre-damage level after about 900 s (15 minutes). In contrast the ECAP observed at electrode E13, which is on a far side of the injury site from the stimulus site, deteriorates sharply at the time of injury, and continues to deteriorate for the entire 15 minute observation period and never recovers. Thus the time course for damage to the cord is relatively rapid when the damage is significant.

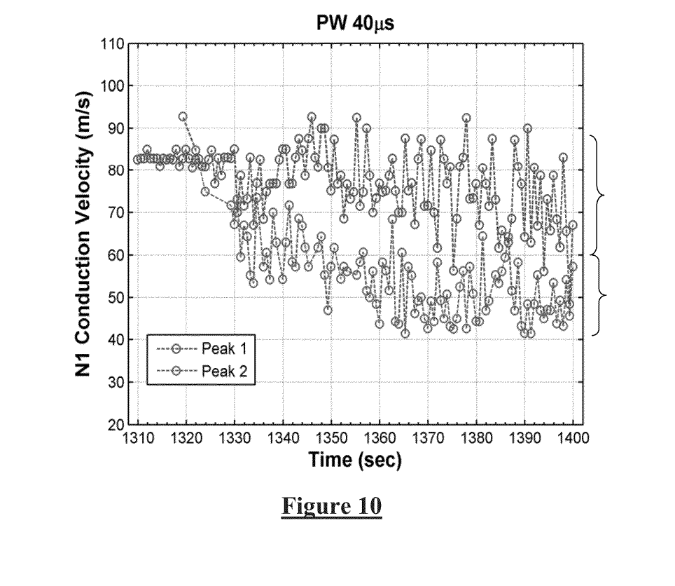

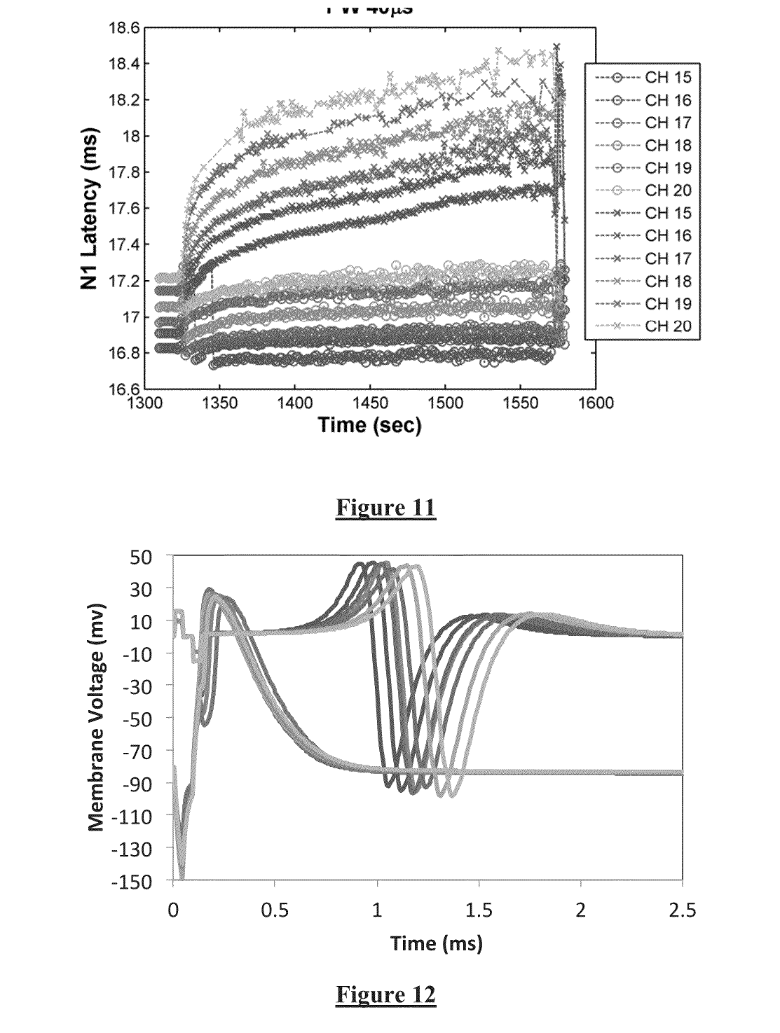

FIG. 10 shows that a reduction in conduction velocity is a further diagnostic of neural damage. FIG. 11 shows that an increase in the latency of the ECAP second peak (ie the N1 peak) is also a diagnostic of neural damage.

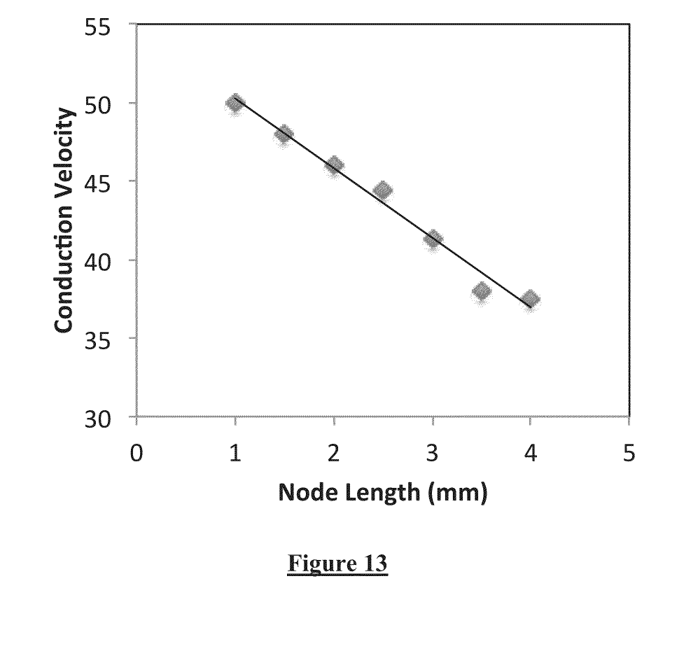

The results of FIGS. 9-11 were compared with computational models of fiber responses and the observed behavior is consistent with modeled behavior where the length of node of Ranvier was increased. FIG. 12 is a computational model of the membrane voltage over time for increasing length of the node of Ranvier, and FIG. 13 is a computational model of the conduction velocity versus the length of the node of Ranvier. In animal studies compression of the spinal cord has been reported and was found to induce acute demyelination and exposure of K+ channels. The compressive injury induces a paranodal retraction which increases the widths of the nodes of Ranvier and leads to exposure of potassium channels. Electrical stimulation has also been shown to produce paranodal retraction and as such the technique described is also applicable for assessment of damage due to electrical stimulation parameters. Another mode of neural injury may arise for example in the case of multiple sclerosis and the destruction of myelin sheaths of neurons leading to a different time profile and/or amplitude profile of ECAP changes which can thus be distinguished as a unique mode of neural injury.

Thus, the preceding investigations and modelling illustrate that low levels of applied pressure to the spinal cord cause reversible effects that can be continuously monitored and identified. When the pressure reaches a critical level the effects become irreversible, the ECAPs cannot propagate and the effect is readily discernable in real time. These observations thus identify a potential new, easy to use, diagnostic method to sense pressure induced damage in the spinal cord during a lead implant procedure.

Thus ECAPs may be useful in monitoring neural injury. Neural injury during lead implantation as a result of pressure applied to the spinal cord during lead implantation can have serious and long lasting effects and may not be noticed until after the procedure has been performed

We have also observed in an animal model that ECAPs can identify the onset of neural injury. The lack of quantitative markers for pain has made the evaluation of new treatments difficult. We have presented data that shows that the ECAP can be used as a biomarker not only for the evaluation of pressure induced damage to the spinal cord but may also be able to be used to identify different pain conditions. Electrical stimulation of the dorsal columns activates a population of neurons of various sizes and properties. These signals are then recorded and make up the ECAP signal. Data from sheep showed that pressure induced ischemic effects on the spinal cord can result in permanent damage to selected neurons. This was confirmed with histological analysis. Changes in ECAP properties similar to those seen in the sheep were also found in patients with chronic pain. Damage and disease can alter ECAP signals by selectively knocking out different neuron populations. These effects can be seen in the properties of the ECAPs.

ECAPs may thus be used to monitor pressure and evaluate its effects on damage to the spinal cord during lead placement. Low levels of applied pressure to the spinal cord cause reversible effects that can be continuously monitored and identified and remedial surgical action taken. The effect of irreversible damage caused by increased pressure can be discernible in real time. Compared to other monitoring techniques, ECAPs promise a simple easy to interpret diagnostic method to sense pressure induced damage in the spinal cord during lead implantation.

Pressure and mechanical manipulation represent only a single source of possible injury, which may occur to the spinal cord. Natural processes such as disease may also damage spinal cord, for instance demyelination due to multiple sclerosis or other neuro-degenerative disease. The techniques described above are applicable to assessment of damage from these sources as well as direct damage due to surgical intervention.

The present invention recognises that using the direct measurement of compound action potentials in the dorsal columns in response to electrical stimulation provides improved insight into the properties of these nerves in both large animals and patients with chronic pain.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not limiting or restrictive.

1. A method of monitoring for neural injury. the method comprising:
delivering a stream of control stimuli to a neural pathway over time;
measuring neural responses evoked by the control stimuli over time;
monitoring a diagnostic parameter of the measured neural responses over time, in order to detect a change in the diagnostic parameter, and
if a change in the diagnostic parameter occurs over time, outputting an indication that neural injury has occurred.

2. The method of claim 1 when used to provide an essentially immediate diagnostic to rapidly detect the onset of the effects of neural injury.

3. The method of claim 1 or claim 2 wherein the control stimuli are delivered, and the diagnostic ECAP parameter is monitored, substantially continuously.

4. The method of any one of claims 1 to 3 when performed throughout a surgical procedure.

5. The method of any one of claims 1 to 3 when performed throughout a postoperative period of interest.

6. The method of any one of claims 1 to 5 wherein the diagnostic parameter comprises ECAP amplitude, and wherein a change in ECAP amplitude is taken as a diagnostic indicator of neural injury.

7. The method of any one of claims 1 to 6 wherein the diagnostic parameter comprises ECAP conduction velocity, and whereby a change in conduction velocity is taken as a diagnostic indicator of neural injury.

8. The method of any one of claims 1 to 7 wherein the diagnostic parameter comprises ECAP peak latency, whereby a change in ECAP peak latency is taken as a diagnostic indicator of neural injury.

9. The method of any one of claims 1 to 8 further comprising observing the diagnostic indicator over multiple stimulus-measurement cycles, so that a curve of the diagnostic indicator can be identified from multiple measurement points.

10. The method of any one of claims 1 to 9 when performed during implantation of an electrode lead during surgery, whereby electrodes of the electrode lead being implanted serve as both the stimulus and sense electrodes, so as to continuously intraoperatively monitor the spinal cord or nerve for damage.

11. The method of any one of claims 1 to 10 wherein a plurality of diagnostic indicators are monitored and the indication that a neural injury has occurred contains an indication of a likely mode of injury.

12. An implantable device for monitoring for neural injury, the device comprising:
a plurality of electrodes including one or more nominal stimulus electrodes and one or more nominal sense electrodes;
a stimulus source for providing a stimulus to be delivered from the one or more stimulus electrodes to a neural pathway in order to give rise to an evoked action potential on the neural pathway;
measurement circuitry for recording a neural compound action potential signal sensed at the one or more sense electrodes; and
a control unit configured to:
deliver a stream of control stimuli to the neural pathway over time;
measure neural responses evoked by the control stimuli over time;
monitor a diagnostic parameter of the measured neural responses over time, in order to detect a change in the diagnostic parameter, and
if a change in the diagnostic parameter occurs over time, output an indication that neural injury has occurred.

13. A non-transitory computer readable medium for monitoring for neural injury, comprising the following instructions for execution by one or more processors:
computer program code means for delivering a stream of control stimuli to a neural pathway over time;
computer program code means for measuring neural responses evoked by the control stimuli over time;
computer program code means for monitoring a diagnostic parameter of the measured neural responses over time in order to detect a change in the diagnostic parameter, and
computer program code means for, if a change in the diagnostic parameter occurs over time, outputting an indication that neural injury has occurred.
